(19) United States
(12) Patent Application Publication
(10) Pub. No.: US 2014/0350094 A1
(43) Pub. Date: Nov. 27, 2014

(54) SOLID COMPOSITION CONTAINING CATECHINS

(71) Applicants: UMEKEN CO., LTD., Osaka (JP); KOWA COMPANY, LTD., Aichi (JP)

(72) Inventors: Kazuhiro Tanie, Osaka (JP); Mikio Yokoyama, Tokyo (JP); Toru Yuasa, Tokyo (JP)

(73) Assignees: UMEKEN CO., LTD., Osaka (JP); KOWA COMPANY, LTD., Aichi (JP)

(21) Appl. No.: 14/284,779

(22) Filed: May 22, 2014

(30) Foreign Application Priority Data
May 27, 2013 (JP) 2013-110975

(51) Int. Cl. A61K 31/353; A61K 47/10

(52) U.S. Cl. CPC A61K 31/353; A61K 47/10
USPC 514/456

(57) ABSTRACT

A solid composition which contains a large amount of catechins and has solved problems concerning quality and producibility for exerting useful action and effects derived from catechins. The solid composition contains catechins and glycerol.

This application claims priority to Japanese Patent Application No. 2013-110975, filed May 27, 2013, the entire contents of which are hereby incorporated by reference.

### BACKGROUND OF THE INVENTION

1. Field of the Invention

The present invention relates to a solid composition containing catechins.

2. Description of the Related Art

The number of people who prefer oriental medicine for treatment and prevention of chronic or long-term diseases is increasing in so-called advanced countries, which have long been aging societies. In oriental medicine, crude drugs were conventionally taken as infusions and decoctions. Such dosage forms, however, require troublesome preparation when taken. For this reason, in recent years, crude drug extracts which are easy to take have been put into circulation and frequently used. Among the extracts, in particular, solidified extracts such as extract granules and extract powder are widely used. As preparations using solidified extracts, tablets and pills are used because of easy handling.

In Japan, as compared with tablets, pills are persistently adopted from the viewpoints of handling and taking properties and familiarness which for example comes from traditional use of pills.

As described in Japanese Patent Laid-open Publication No. 2007-159541, catechins, which are abundantly contained in tea leaves of green tea, are known to have excellent action and effects, such as removing active oxygen, suppressing absorption of sugar, suppressing an increase in blood pressure, burning fat, anticancer, improving liver function, antioxidation, antibacterial, beautifying the skin, deodorization, antiallergy, suppressing cholesterol absorption, and preventing cerebral infarction. A high intake of catechins is however necessarily to cause catechins to exert their excellent action and effects, and it is said that an adult needs to drink not less than 10 cups of green tea per day. Therefore, there is a desire for the development of a composition which enables catechins to be taken easily and in a large amount. Japanese Patent Laid-open Publication No. 2007-159541 proposes a liquid which contains catechins at a high concentration.

### SUMMARY OF THE INVENTION

### Technical Problem

Still, it is rather difficult to drink 10 cups of green tea. Thus, an object of the present invention is to provide a compound which enables a high intake of catechins without drinking a large amount of green tea.

The present inventors conducted intensive studies in preparing a solid composition containing a large amount of catechins and found that the use of water in preparing the solid composition causes the compound to expand, leading to the occurrence of problems affecting the quality.

Thus, the present inventors opted for the use of monohydric alcohol, which turned out to cause viscosity, leading to the occurrence of problems making production of the solid composition difficult.

Accordingly, an object of the present invention is to provide a catechin-rich solid composition which solves the above-described quality problems and can be successfully produced.

### Solution to Problem

Through intensive studies in view of the above, the present inventors found that problems concerning quality and producibility can be solved by using glycerol in preparing a solid composition containing a large amount of catechins. The present invention was thus completed.

### Advantageous Effects of the Invention

The solid composition of the present invention has solved problems concerning quality and producibility and is capable of containing a large amount of catechins and exerting useful action and effects derived from the catechins.

The foregoing and other objects, features, aspects and advantages of the present invention will become more apparent from the following detailed description of the present invention.

### DETAILED DESCRIPTION OF THE PREFERRED EMBODIMENTS

The solid composition of the present invention contains a large amount of catechins therein. The solid composition of the present invention will be hereinafter described.

### <Catechins>

Catechins contained in the solid composition of the present invention are not particularly limited, and examples of the catechins include catechin, gallocatechin, catechin gallate, gallocatechin gallate, epicatechin, epigallocatechin, epicatechin gallate, epigallocatechin gallate, and the like. The catechins can be produced through known methods, or commercially available catechins can be used.

Catechins are a type of polyphenol and known to be present in tea leaves (Camellia sinensis) of green tea, black tea, oolong tea, and the like. Catechins in the present invention encompass tea leaves themselves and extracts of tea leaves as well. Tea leaves preferred in the present invention are tea leaves of green tea.

The tea leaves can be in a form adjusted as necessary, and may be cut or crushed into small pieces or small masses or pulverized into powder for use. Examples of the tea leaf extracts include an infused liquid obtained based on known methods described in General Rules for Preparations, The Japanese Pharmacopoeia, Sixteenth Edition by suitably sizing the tea leaves and adding an infusing agent to the sized tea leaves, and a concentrated liquid of the infused liquid, such as so-called “extract” and “tincture”. Also can be used is a “dried extract” or the like which is obtained by drying the foregoing into a shape of solid-mass, granule, powder, or the like, that is, by drying a liquid which has been infused with tea leaves.

It is noted that although tea leaf extracts can be prepared based on the above-described known methods, commercially available tea leaf extracts can also be used.

Examples of the above-mentioned infusing agent include: lower monohydric alcohols (for example with 1 to 7 carbon atoms) such as methanol, ethanol, and n-butanol; lower polyhydric alcohols (for example with 1 to 7 carbon atoms) such as ethylene glycol, propylene glycol, 1,3-butylene glycol, and glycerol; ethers such as diethyl ether; ketones such as acetone and ethyl methyl ketone; esters such as acetic acid ethyl ester; halogenoalkanes such as dichloromethane and chloroform; aromatic hydrocarbons such as benzene and toluene; water; and the like. The foregoing may be used alone or as a mixture of two or more. Preferred among the foregoing are lower monohydric alcohols, ethers, ketones, their mixtures with water, and water alone, more preferred are ethanol, acetone, diethyl ether, their mixtures with water, and water alone, and particularly preferred is hydrous ethanol.

Although a content of catechins in the solid composition of the present invention may be determined by examining a daily dosage as appropriate according to, for example, the sex, age, and symptoms of a taker and the administration method of the solid composition, the content preferably allows a daily dosage of 0.05 to 2 g, more preferably a daily dosage of 0.1 to 1.5 g, and even more preferably a daily dosage of 0.15 to 1 g. It is noted that the content is not limited to the aforementioned contents, and can be increased or decreased as appropriate according to the sex, age, symptoms, and the like.

Further, when the intended action of catechins is for example suppression of sugar absorption, a content which allows a daily dosage of 600 to 800 mg is preferred. When fat burning action is intended, a content which allows a daily dosage of 400 to 600 mg is preferred. When the intended action is improving liver function, a content which allows a daily dosage of 50 to 500 mg is preferred. When antioxidation action is intended, a content which allows a daily dosage of 200 to 300 mg is preferred. When the intended action is suppressing cholesterol absorption, a content which allows a daily dosage of 350 to 500 mg is preferred. When the intended action is preventing cerebral infarction, a content which allows a daily dosage of 300 to 400 mg is preferred.

Still further, the content of catechins with respect to the total mass of the solid composition of the present invention is preferably 5 to 80% by mass, more preferably 10 to 60% by mass, and even more preferably 15 to 50% by mass.

### <Glycerol>

Glycerol contained in the solid composition of the present invention is a known compound having a chemical name, 1,2,3-propanetriol.

The content of glycerol may be examined as appropriate according to a content of catechins in the solid composition, and is preferably 0.5 to 20% by mass, more preferably 1 to 15% by mass, and even more preferably 2 to 10% by mass, with respect to the total mass of the solid composition. The mass ratio of catechins to glycerol (catechins: glycerol) in the solid composition is preferably 1:1 to 10:1, more preferably 2:1 to 8:1, and even more preferably 2:1 to 5:1.

Glycerol can be produced by known methods or commercially available glycerol can be used. For example, “glycerol” or “concentrated glycerol” as described in The Manual of The Japanese Pharmacopoeia, Sixteenth Edition can be used.

The method for producing the solid composition of the present invention is not particularly limited. For example, the solid composition can be prepared according to known tablet preparation methods, granulation methods, and pill preparation methods which are described in General Rules for Preparations, The Japanese Pharmacopoeia, Sixteenth Edition and the like. The solid composition of the present invention is not particularly limited in its dosage form and can be in various dosage forms such as granules, pills, tablets, powders, and capsules; however, granules and pills are preferred when the management of dosage of catechins and the size of dosage form are taken into consideration.

The solid composition of the present invention can include various components which attribute to health as long as the components do not inhibit action derived from catechins.

Examples of such components include: vegetables such as sweet potato, carrot, cabbage, spinach, lotus root, bok Choy (Brassica rapa var. chinensis), Chinese cabbage (Brassica rapa var. pekinensis), greater burdock (Arctium lappa), broccoli, asparagus, bean sprouts, yam, lettuce, taro, enokitake mushroom (Flammulina velutipes), maitake mashroom (Grifola frondosa), Japanese mustard spinach (Brassica rapa var. perviridis), garland chrysanthemum, buna shimeji mushroom (Hypsizygus marmoreus), red leaf lettuce, and turnip and a fermentation product thereof; rice such as unpolished rice, polished rice with germ, rice bran, red rice, and black rice and a fermentation product thereof; bacteria such as lactic acid bacteria, Lactobacillus, Bifidobacterium, and Bacillus subtilis var. natto and an extract thereof; and the like.

In preparing the solid composition of the present invention, formulation additives can be used. Examples of the formulation additives include various physiologically acceptable additives, such as excipients, binders, lubricants, pH adjusters, colorants, sweetener, disintegrators, and the like.

Examples of the excipient include powdered glycyrrhiza, a crude glycyrrhiza extract, starches, dried yeasts, yeast extracts, purified lanolin, crystalline cellulose, lactose, sucrose, mannitol, and the like.

Examples of the binder include hydroxypropylmethylcellulose, hydroxypropylcellulose, gelatin, pregelatinized starches, polyvinylpyrrolidone, polyvinyl alcohol, pullulan, and the like.

Examples of the lubricant include magnesium stearate, talc, and the like.

Examples of the pH adjuster include: citric acid, malic acid, lactic acid, tartaric acid, acetic acid, hydrochloric acid, phosphoric acid, and a salt thereof; sodium hydroxide, potassium hydroxide, magnesium hydroxide, calcium hydroxide; and the like.

Examples of the colorant include titanium oxide, Food Yellow No. 4, Food Yellow No.5, iron sesquioxide, yellow iron sesquioxide, Food Red No. 3, and the like.

Examples of the sweetener include white soft sugar, liquid sugar, fructose, fructose glucose liquid sugar, reduced maltose syrup, honey, caramel, sorbitol, maltitol, xylitol, erythritol, sucralose, stevia, glycyrrhizic acid or a salt thereof, aspartame, acesulfame potassium, and the like.

Examples of the disintegrator include carmellose, carmellose calcium, croscarmellose sodium, crospovidone, low-substituted hydroxypropylcellulose, and the like.

The solid composition of the present invention contains catechins and therefore can be used, on the basis of action derived from the catechins, as a pharmaceutical, a food for specified health uses, a food with nutrient function claims, and a health food (nutritional supplement, health supplement, dietary supplement, and the like) for prevention and/or treatment of various illness and diseases.

### EXAMPLES

The present invention will be hereinafter described in detail with examples. The present invention is however not limited by these examples at all.

### Example 1

### Production of Pills with a High Catechin Content

The following were mixed and kneaded to provide a wet mass having an appropriate consistency: a green tea extract as catechins in such an appropriate amount as to be 500 parts by mass, a fermentation product of vegetables, cyclodextrin, malted unpolished rice, 321 parts by mass of a blend of Bacillus coagulans and Lactobacillus brevis sp. Coagulans, an appropriate amount of sweet potato (cultivar: Ayamurasaki) powder, starch, defatted rice grain, citrus fiber, and 135 parts by mass of glycerol.

The obtained wet mass was shaped into a spherical form to provide granular materials, which were dried to provide pills having a diameter of about 7.5 mm. Seven pills containing 500 mg of catechins were obtained.

Even after storage at 50° C. for 1 month, the obtained pills had no change in appearance and were stable. Further, since the pills of Example 1 include almost no water, the strains included in the pills have a high viability.

### Comparative Example 1

### Production of Pills with a High Catechin Content and No Glycerol Content (1)

Mixing was started in the same manner as in Example 1 except that glycerol was replaced with ethanol; however, viscosity was exhibited, and appropriate mixing could not be achieved. Consequently, a wet mass having an appropriate consistency could not be suitably prepared from the view point of industrial production.

### Comparative Example 2

### Production of Pills With a High Catechin Content and No Glycerol Content (2)

Pills were obtained in the same manner as in Example 1 except that glycerol was replaced with water. The obtained pills were stored at 50° C. Two weeks later, the stored pills had swelled and it turned out that pills containing catechins for which water has been used are unstable. Further, since the pills of Comparative Example 2 include water, the strains included in the pills have a low viability.

### INDUSTRIAL APPLICABILITY

The present invention makes it possible to successfully produce a high-quality solid composition which contains a large amount of catechins exhibiting useful pharmacological action.

Although the present invention has been described in detail, it is clearly understood that the same is by way of illustration and example only and is not to be taken by way of limitation, the scope of the present invention being interpreted by the terms of the appended claims.

What is claimed is:

1. A solid composition comprising catechins and glycerol.

2. The solid composition according to claim 1, wherein the catechins are contained at 15 to 50% by mass in the solid composition.

3. The solid composition according to claim 1, wherein the solid composition is in a granule or pill dosage form.

4. The solid composition according to claim 2, wherein the solid composition is in a granule or pill dosage form.
